(19) United States
(12) Patent Application Publication
(10) Pub. No.: US 2002/0097155 A1
(43) Pub. Date: Jul. 25, 2002

(54) Combination breathing monitor alarm and audio baby alarm

(76) Inventors: Cynthia L. Cassel, Connellsville, PA (US); Robert H. Cassel JR., Connellsville, PA (US)

Correspondence Address: John D. Gugliotta, P.E., Esq., 202 Delaware Building, 137 South Main Street, Akron, OH, 44308, US

(21) Appl. No.: 09/767,413

(22) Filed: Jan. 23, 2001

(51) Int. Cl. G08B 23/00; A61B 5/08

(52) U.S. Cl. 340/573.1; 340/575; 340/573.7; 600/390; 600/532; 600/534

(57) ABSTRACT

A combination breathing monitor alarm and audio baby alarm is provided that includes an attachable transmitter forming a main body of a linearly elongated, pliable chest strap of a soft and formable material that is easily wrapable about the chest of an infant. A receiver housing receiver control circuitry receiving signals transmitted by the transmitter.

### RELATED APPLICATIONS

The present invention was first described in Disclosure Document Number 479,636 filed on Sep. 11, 2000. There are no previously filed, nor currently any co-pending applications, anywhere in the world.

### BACKGROUND OF THE INVENTION

1. Field of the Invention

The present invention relates generally to a respiratory detection devices and, more particularly, to a combination breathing monitor alarm and otherwise conventional audio baby alarm.

2. Description of the Related Art

As any parent will attest, the safety and well-being of their children is of the utmost importance and is by far their primary concern. Unsurprisingly, there are a wide variety of products aimed at providing a safe home environment for children by preventing the accidents and mishaps that occur all too often. However, one event that strikes fear into the hearts of parents and care providers everywhere is that of Sudden Infant Death Syndrome (S.I.D.S.). Although the occurrence of S.I.D.S. is of very low probability, it occurs without warning and the outcome is almost always devastating and heartbreaking. Other than a parent or care giver co4nstantly watching the infant while he or she sleeps, there is little they can do.

A search of the prior art did not disclose any patents that read directly on the claims of the instant invention; however, the following references were considered related.

The following patents disclose a motion and sound monitor and simulator device to prevent SIDS:

U.S. Pat. No. 6,011,477 issued in the name of Teodorescu et al.

U.S. Pat. No. 5,986,549 issued in the name of Teodorescu

U.S. Pat. No. 5,684,460 issued in the name of Scanlon

U.S. Pat. No. 5,515,865 issued in the name of Scanlon

U.S. Pat. No. 4,679,036 issued in the name of Cheng

U.S. Pat. No. 5,241,300 issued in the name of Buschmann describes a trans-illuminated optical fiber for monitoring infant's breathing pattern.

U.S. Pat. No. 4,851,816 issued in the name of Macias et al. discloses an apparatus for specific fluid detection for preventing SIDS.

U.S. Pat. No. 4,146,885 issued in the name of Lawson, Jr. describes a flexible membrane for detecting an infant's breathing pattern.

And, U.S. Pat. No. 6,043,747 issued in the name of Altenhofen, describes a baby monitor two-way audible communication device.

Consequently, there is a need for a means by sleeping infants can be constantly monitored and protected against the occurrence of S.I.D.S.

### SUMMARY OF THE INVENTION

It is therefore an object of the present invention to provide an improved respiratory detection device.

It is a feature of the present invention to provide an improved respiratory detection device that incorporates an audible transmission monitoring device.

Briefly described according to one embodiment of the present invention, an apparatus is provided that monitors sleeping for the presence of breathing. The invention sounds an audible alarm if breathing is not detected on a regular basis, thus preventing the occurrence of Sudden Infant Death Syndrome. The first component of the invention is an elastic belt that is placed around an infant's chest and is fastened together with a hook and loop fastener such as Velcro®. Located on this belt is a sensor, similar to a microphone, that detects the regular breathing patterns of the infant. A transmitter, powered by watch batteries, transmits the sensor signals to a nearby receiver located in the same room as the infant. The receiver processes the signals, via an algorithmic sequence, looking for the presence of stopped breathing. In the event the infant stops breathing, an audible alarm sounds. If the infant then resumes breathing on their own, the alarm will stop. If the infant still does not resume breathing, the audible alarm remains sounding to alert the parent or care giver that immediate attention, such as the administration of CPR is required.

The use of the present invention allows parents and care providers to ensure that sleeping infants are protected from the occurrence of Sudden Infant Death Syndrome, in a manner which is quick, safe and effective.

An additional advantage of the present invention is that it provides an integral audio monitoring device for transmitting audio signals in a wireless manner to a remote monitoring receiver.

### BRIEF DESCRIPTION OF THE DRAWINGS

The advantages and features of the present invention will become better understood with reference to the following more detailed description and claims taken in conjunction with the accompanying drawings, in which like elements are identified with like symbols, and in which:

FIG. 1 is a perspective view of a combination breathing monitor alarm and audio baby alarm according to the preferred embodiment of the present invention;

FIG. 2 is a side elevational view thereof; and

FIG. 3 is an electrical schematic for the control circuitry for use therewith.

### DESCRIPTION OF THE PREFERRED EMBODIMENTS

The best mode for carrying out the invention is presented in terms of its preferred embodiment, herein depicted within the Figures.

1. Detailed Description of the Figures

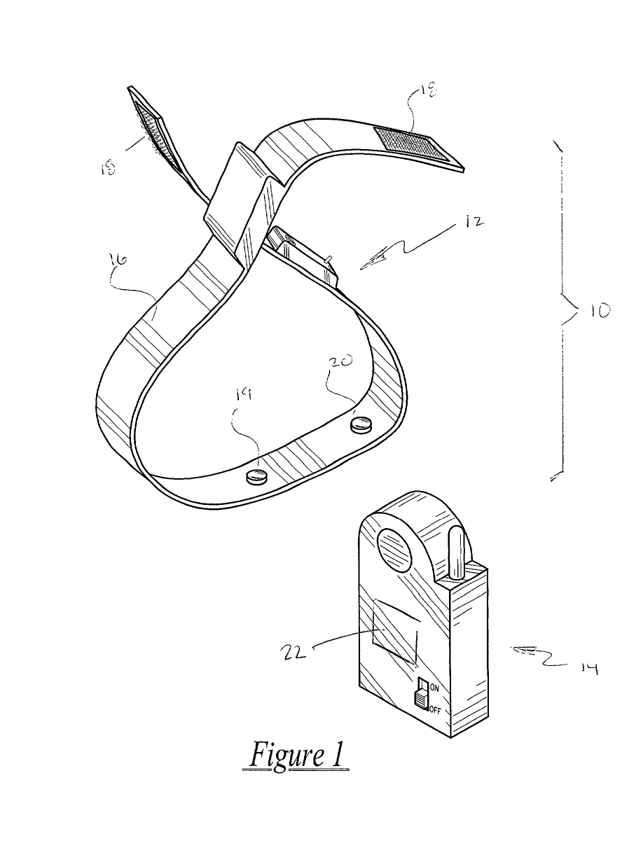

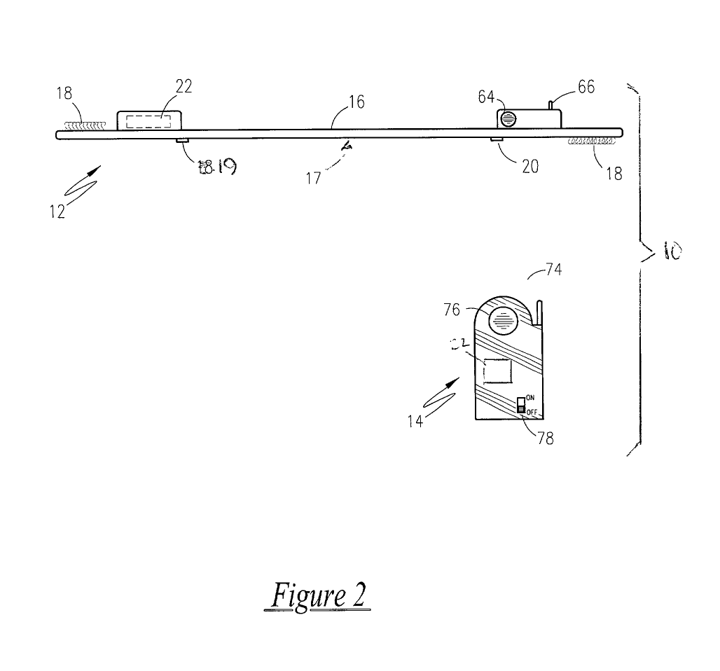

Referring now to FIG. 1-2, a combination breathing monitor alarm and audio baby alarm 10 is shown, according to the present invention, including an attachable transmitter 12 in combination with a receiver 14. The transmitter 12 forms a main body of a linearly elongated, pliable chest strap 16 having hook and loop fastener means 18 supported at each linear end. It is anticipated that the chest strap 16 would be soft and formable, and easily wrapable about the chest of an infant in a manner that would be safe and comfortable. In this manner, the hook and loop fastener means 18 can allow for the chest strap 16 to be connected in a manner circumscribing the wearers chest. The chest strap 16 has a flat, smooth inner surface 17 supporting a first resonant sensor 19 spaced laterally apart from a second resonant sensor 20. A microphone 64 is further housed in the chest strap 16, and communicates with transmitter control circuitry housed therein, whose function will be described in greater detail below. Further, a battery housing 22 for securely storing batteries in a removable manner is provided within the chest strap 16 for providing portable electrical power for powering the transmitter control circuitry.

A receiver 14 is provided housing receiver control circuitry, as will be described in greater detail below, for receiving signals transmitted by the transmitter 12. It is anticipated that the receiver 14 will be used physically remotely from the transmitter 12, and would thereby be in wireless radio communication with the transmitter 12. It is further anticipated that a lighting means 22, shown herein as an incandescent illumination panel, would provide the functionality of a conventional “night-light” as well.

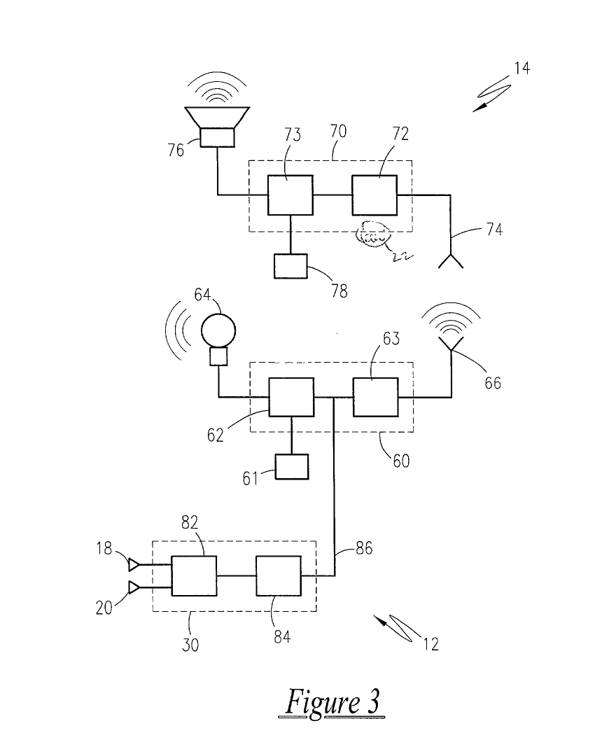

FIG. 3 shows in greater detail the transmitter control circuitry and receiver control circuitry. The transmitter control circuitry has a transmitter control central processing unit 60 including a conventional radio frequency transmitter 63 communicating with an antenna 66 and controlled by a conventional analog to digital microphone amplification circuit 62 in communication with a microphone 64. An on/off switch 61 controls the input of electrical power to both circuits 62, 63. A receiver control central processing unit 70 including a conventional radio frequency receiver 72 communicating with an antenna 74 and controlled by a conventional digital to analog speaker amplification circuit 73 in communication with a speaker 76. An on/off switch 78 controls the input of electrical power to both circuits 72, 73.

The transmitter 12 further incorporates a respiration monitor 30 for monitoring the respiration of the user as well as interacting with the transmitter control circuitry for transmitting a respiration alarm signal. The first resonant sensor 18 and second resonant sensor 20 are anticipated as being in physical contact with the chest of an infant. It is anticipated that the first resonant sensor 18 detects respiration and/or movement of the infant, while the second resonant sensor 20 detects heart rate or pulse. Such redundancy will allow for prevention of “false” alarming should the infant move during sleep in a manner that prevents adequate communication with the sensors 18, 20. A signal processor 82 compares the respiration related signal pattern to a stored pattern, and monitors the heart rate or pulse as compared with an initial baseline measurement. A comparitor circuit 84 determines if either of the measured characteristic fall below an alarm point, and generate an alarm output impulse 86 that communicates with the conventional radio frequency transmitter 63, forming an synthesized signal that communicating with an antenna 66 and results in an alarm or annunciation signal of a predetermined frequency for audible transmission through the speaker 76 of the receiver 14. In this manner, both normal, monitoring sounds as well as the incidental alarm annunciator can be transmitted via the same transmitter/receiver combination.

2. Operation of the Preferred Embodiment

In operation, the combination breathing monitor alarm and audio baby alarm is to monitor the sounds within the baby's environment and transmit those sounds for reproduction to the receiver. The chest strap 16 is placed on or around the infant for providing audible, respiratory, and circulatory monitoring. The care giver can then monitor for the detection of audibly produced distress type sounds. Concurrently, should the respiration monitor 30 identify alarmable events in the infant's breathing or heart rate, an alarm annunciator signal is transmitted for reproduction to the same receiver. In this manner the care giver can be notified immediately of a distress event of the type that would not necessarily be accompanied by an audible distress type sound.

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The embodiments were chosen and described in order to best explain the principles of the invention and its practical application, to thereby enable others skilled in the art to best utilize the invention and various embodiments with various modifications as are suited to the particular use contemplated. It is intended that the scope of the invention be defined by the Claims appended hereto and their equivalents. Therefore, the scope of the invention is to be limited only by the following claims.

What is claimed is:

1. A combination breathing monitor alarm and audio baby alarm comprising:
an attachable transmitter forming a main body of a linearly elongated, pliable chest strap of a soft and formable material that is easily wrapable about the chest of an infant; and
a receiver housing receiver control circuitry for receiving signals transmitted by said transmitter.

2. The combination of claim 1, wherein said transmitter further comprises a hook and loop fastener means to allow for the chest strap to be connected in a manner circumscribing the wearers chest.

3. The combination of claim 1, wherein said chest strap 16 has a flat, smooth inner surface supporting a first resonant sensor spaced laterally apart from a second resonant sensor and a microphone housed with said chest strap which communicates with transmitter control circuitry housed therein.

4. The combination of claim 1, wherein said receiver is in wireless radio communication with said transmitter.

5. The combination of claim 1, wherein said receiver comprises a lighting means.

6. The combination of claim 3, wherein said transmitter control circuitry has a transmitter control central processing unit including a conventional radio frequency transmitter communicating with an antenna and controlled by an analog to digital microphone amplification circuit in communication with a microphone.

7. The combination of claim 1, wherein said receiver control circuitry comprises a receiver control central processing unit including a conventional radio frequency receiver communicating with an antenna and controlled by a digital to analog speaker amplification circuit in communication with a speaker.

8. The combination of claim 6, wherein said transmitter further incorporates a respiration monitor for monitoring the respiration of the user as well as interacting with the transmitter control circuitry for transmitting a respiration alarm signal.

9. The combination of claim 8, wherein said respiration monitor comprises a first resonant sensor for detecting respiration and movement of the infant a second resonant sensor for detecting heart rate and pulse.

10. The combination of claim 9, wherein said respiration monitor further comprises a signal processor that compares the respiration related signal pattern to a stored pattern, and monitors the heart rate or pulse as compared with an initial baseline measurement.

11. The combination of claim 10, wherein said respiration monitor comprises a comparitor circuit that determines if either of the measured characteristic fall below an alarm point, and generate an alarm output impulse that communicates with the radio frequency transmitter, forming an synthesized signal that communicating with an antenna and results in an alarm or annunciation signal of a predetermined frequency for audible transmission through said speaker of said receiver.
